(19) United States
(12) Patent Application Publication
(10) Pub. No.: US 2017/0059569 A1
(43) Pub. Date: Mar. 2, 2017

(54) METHODS AND COMPOSITIONS FOR CYTOMETRIC DETECTION OF CIRCULATING TUMOR CELL MARKERS IN A SAMPLE

(71) Applicant: Becton, Dickinson and Company, Franklin Lakes, NJ (US)

(72) Inventor: Margaret Inokuma, San Jose, CA (US)

(21) Appl. No.: 15/236,278

(22) Filed: Aug. 12, 2016

Related U.S. Application Data
(60) Provisional application No. 62/210,791, filed on Aug. 27, 2015.

(51) Int. Cl. G01N 33/574; G01N 33/58; G01N 15/14

(52) U.S. Cl. CPC G01N 33/574; G01N 15/14; G01N 33/582; G01N 2333/43521; G01N 2015/0065

(57) ABSTRACT

Methods of determining whether a circulating tumor cell (CTC) marker, e.g., a CTC or fragment thereof, is present in a sample, such as a blood sample, are provided. Aspects of the methods include flow cytometrically assaying a fluorescently labeled sample that has been fluorescently labeled with a fluorescently labeled chlorotoxin binding member to determine whether a CTC marker is present in the sample. Also provided are compositions for practicing the methods. The methods and compositions find use in a variety of applications.

### CROSS-REFERENCE TO RELATED APPLICATION

Pursuant to 35 U.S.C. §119 (e), this application claims priority to the filing date of U.S. Provisional Patent Application Ser. No. 62/210,791, filed Aug. 27, 2015, the disclosure of which application is incorporated herein by reference.

### INTRODUCTION

Circulating tumor cells (CTCs) are cells shed from tumors that enter into a subject's blood stream (Rodic et al., “Detection methods of circulating tumor cells in cutaneous melanoma: a systematic review,” Critical Reviews in Oncology/Hematology (2014) 91.1, 74-92). Once in the blood, these cells can circulate through the subject's body, where they can invade other tissues and grow new tumors (Rodic et al., 2014). CTCs are thus implicated in metastasis, which is the primary cause of death in subjects with cancer (Lowes et al., “Adaptation of semiautomated circulating tumor cell (CTC) assay for clinical and preclinical research applications,” Journal of Visualized Experiments (2014) 84, e51248).

Early detection and monitoring of tumor growth is critical for the successful treatment of cancer patients. However, detecting metastases is difficult because a metastasis can be formed beginning with a single cell shed off by a primary tumor. In addition, CTCs largely vary in their morphology and are a rare presence in the blood. Current imaging techniques such as X-ray, PET-scan, and CT-scans cannot provide early diagnosis because of insufficient sensitivity. Successful detection of CTCs requires a technique that identifies and isolates them without harm to the cells.

Many techniques currently applied to isolate and characterize CTCs use antibody based positive selection. One employed technique is magnetic-activated cell sorting, such as the CellSearch method (Lopez-Riquelme et al., “Imaging cytometry for counting circulating tumor cells: comparative analysis of the CellSearch vs ImageStream systems,” APMIS (2013) 121.12, 1139-43). This method involves taking a blood sample from the patient and incubating the cells with magnetic nanoparticles coated with antibodies against a particular surface antigen. The CellSearch method identifies cells expressing several antigens commonly associated with cancer, EpCAM and Cytokeratin 8, 18, or 19, and is used to detect various types of metastatic cancers including but not limited to prostate, breast, lung, colorectal, ovarian, pancreatic, and bile duct cancer (Allard et al., “Tumor cells circulate in the peripheral blood of all major carcinomas but not in healthy subjects or patients with nonmalignant diseases,” Clinical Cancer Research (2004) 10.20, 6897-904), (Yang et al., “Circulating tumor cells are associated with poor overall survival in patients with cholangiocarcinoma,” Hepatology (2015) 10.1002/hep.27944). Cells expressing the antigen bind to the nanoparticle and then are isolated by application of a magnetic field. A study done with metastatic breast cancer patients found that the CellSearch method and a similar test called the AdnaTest had detection rates of 36% and 22%, respectively (Van der Auwera et al., “Circulating tumour cell detection: a direct comparison between the CellSearch System, the AdnaTest and CK-19/mammaglobin TR-PCR in patients with metastatic breast cancer,” British Journal of Cancer (2010) 102.2, 276-84). Generally, the detection rate of CTCs using the CellSearch method is only about 20-25% for patients with primary breast cancer (Onstenk et al., “Improved Circulating Tumor Cell Detection by a Combined EpCAM and MCAM CellSearch Enrichment Approach in Patients with Breast Cancer Undergoing Neoadjuvant Chemotherapy,” Molecular Cancer Therapeutics (2015) 14.3, 821-7).

### SUMMARY

Methods of determining whether a circulating tumor cell (CTC) marker, e.g., a CTC or fragment thereof, is present in a sample, such as a blood sample, are provided. Aspects of the methods include flow cytometrically assaying a fluorescently labeled sample that has been fluorescently labeled with a fluorescently labeled chlorotoxin binding member to determine whether a CTC marker is present in the sample. Also provided are compositions for practicing the methods. The methods and compositions find use in a variety of applications.

### DETAILED DESCRIPTION

Methods of determining whether a circulating tumor cell (CTC) marker, e.g., a CTC or fragment thereof, is present in a sample, such as a blood sample, are provided. Aspects of the methods include flow cytometrically assaying a fluorescently labeled sample that has been fluorescently labeled with a fluorescently labeled chlorotoxin binding member to determine whether a CTC marker is present in the sample. Also provided are compositions for practicing the methods. The methods and compositions find use in a variety of applications.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various embodiments of the invention, aspects of the methods will be reviewed first, followed by a review of systems and kits that find use in practicing embodiments of the methods.

### Methods

As described above, aspects of the invention include methods of determining whether a circulating tumor cell (CTC) marker is present in a sample. By “determining,” it is meant detecting the presence of a CTC marker in the sample being assayed. “Detecting the presence of” includes making a decision based on data obtained via the methods that a CTC marker is present in the sample being assayed. The phrase “CTC marker” refers collectively to CTCs and identifying fragments or portions thereof, e.g., components thereof that may be derived from a CTC. By “CTC,” it is meant a cell of epithelial origin that is present in the circulation of patients, is derived from clones of a primary tumor, and is malignant. In some instances, CTCs that are detected by methods of the invention are CTCs that express MMP2, where in some instances the MMP2 is part of a lipid raft anchored complex containing one or more of MMP2 (Matrix Metalloproteinase 2, e.g., UniProtKB—P08253 (MMP2_HUMAN), MT1-MMP (Matrix Metalloproteinase 14; MMP14, e.g., UniProtKB—P50281 (MMP14_HUMAN), TIMP-2 (Tissue Inhibitor of Metalloproteinase 2, e.g., UniProtKB—P16035 (TIMP2_HUMAN), chloride channels, and other proteins.

Aspects of the methods include flow cytometrically assaying a fluorescently labeled sample that has been fluorescently labeled with a fluorescently labeled chlorotoxin binding member to determine whether a CTC is present in the blood sample. By “fluorescently labeled sample,” it is meant a sample that has been contacted with a fluorescently labeled chlorotoxin binding member, e.g., as described above, such that the sample includes a fluorescently labeled chlorotoxin binding member. The nature of the sample may vary, where types of samples that may be assayed include, but are not limited to physiological samples such blood, tears, lymph fluid, urine, etc. Where the sample is a blood sample, the blood sample may be whole blood or a component or fraction thereof, e.g., plasma, serum, etc., as desired, where in some instances the blood sample is a peripheral blood sample. The blood sample may be one that has been treated in some way to provide for desirable characteristics, e.g., it may have been contacted with an anti-coagulant (e.g., ethylenediaminetetraacetic acid (EDTA), buffered citrate, or heparin), buffer, etc.

In producing the fluorescently labeled blood sample that is assayed in methods of the invention, the blood sample is contacted with a fluorescently labeled chlorotoxin binding member under conditions sufficient for the fluorescently labeled chlorotoxin binding member to bind to CTCs. By “fluorescently labeled chlorotoxin binding member,” it is meant a labeling reagent that includes one or more fluorescent dye moieties stably associated with, e.g., covalently bonded to, a chlorotoxin binding member. By “chlorotoxin binding member,” it is meant a moiety or entity that is chlorotoxin or a functional mimetic thereof. As such, chlorotoxin binding members including moieties, e.g., peptides, that bind to a lipid raft anchored complex containing MMP2, MT1-MMP, TIMP-2, chloride channels, and other proteins.

In some instances, the chlorotoxin binding member is chlorotoxin or a mutant thereof. As such, the chlorotoxin binding member may have a sequence that is identical to the following sequence: MCMPCFTTDHQMARKCDDCCGGKGRGKCYGPQCLCR (SEQ ID NO:01) or be a mutant thereof. Mutants of interest include insertion, deletion and substitution mutants. In some instances, the mutant has a sequence identity that is 60% or greater, such 75% or greater, including 85% or greater, e.g., 90% or greater, to the sequence of SEQ ID NO: 01. Sequence identity may be determined using any convenient protocol, such as the MegAlign, DNAstar (1998) clustal algorithm as described in D. G. Higgins and P. M. Sharp, “Fast and Sensitive multiple Sequence Alignments on a Microcomputer,” (1989) CABIOS, 5: 151-153. (Parameters used are ktuple 1, gap penalty 3, window, 5 and diagonals saved 5).

In addition to the chlorotoxin peptide having the sequence of SEQ ID NO:01, specific chlorotoxin binding members of interest include, but are not limited to, those described in Table 1 of published PCT application publication no. WO/2015/042202; the disclosure of which sequences are herein incorporated by reference. An example of a specific mutant chlorotoxin that may be employed is MCMPCFTTDHQMARRCDDCCGGRGRGKCYGPQCLCR (SEQ ID NO:02). The chlorotoxin binding member may also be a chlorotoxin like peptide. Examples of chlorotoxin like peptides include toxins isolated from the venom of a number of different scorpion species, including but not limited to: Buthus martensii Karsch chlorotoxin (BmKCT), which is a chlorotoxin-like peptide isolated from the venom of the Chinese scorpion indigenous to regions of China and the Korean peninsula and has the sequence CGPCFTTDANMARKCRECCGGIGKCFGPQCLCN (SEQ ID NO: 03); scorpion Androctonus australis (AaCtx) chlorotoxin having the sequence MCIPCFTTNPNMAAKCNACCGSRRGSCRGPQCIC (SEQ ID NO:04), etc. Where the chlorotoxin specific binding member is a chlorotoxin like peptide, it may be the wild type version or a mutant thereof, e.g., as described above. The chlorotoxin binding member domain of the fluorescently labeled chlorotoxin binding members may be a linear or cyclized peptide, as desired.

As summarized above, the fluorescently labeled chlorotoxin binding members employed in methods of the invention include a chlrotoxin binding member domain stably associated with, e.g., covalently bonded to, one or more fluorescent moieties (i.e., fluorescent dye groups). The number of fluorescent moieties stably associated with the chlorotoxin binding member domain may vary, ranging in some instance from 1 to 5, such as 1 to 3, e.g., 1 to 2. In some instances, the fluorescently labeled chlorotoxin binding member includes a single fluorescent moiety covalently bonded to an amino acid residue of the chlorotoxin binding member domain.

The fluorescently labeled chlrotoxin binding members may include any convenient fluorescent dye(s). Fluorescent dyes that may be employed include those having distinct excitation and emission maxima, e.g., where the excitation and emission maxima differ from each other by 50 to 150 nm, such as 75 to 125 nm. In some instances, the dyes have an absorbance maximum ranging from 300 to 700, such as from 350 to 650 and including from 400 to 600 nm, while the emission spectra of the dyes ranges from 400 to 800, such as 425 to 775 and including 450 to 750 nm.

Fluorescent dyes (i.e., labels) that may be employed include, but are not limited to: fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Specific fluorescent labels of interest include, but are not limited to: indocarbocyanine (C3), indodicarbocyanine (C5), Cy3, Cy3.5, Cy5, Cy5.5, Cy7, Texas Red, Pacific Blue, Oregon Green 488, Alexa Fluor 355, Alexa Fluor 488, Alexa Fluor 532, Alexa Fluor 546, Alexa Fluor 555, Alexa Fluor 568, Alexa Fluor 594, Alexa Fluor 647, Alexa Fluor 660, Alexa Fluor 680, Alexa Fluor 700, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like.

In some instances, the polymeric dye is a fluorescent polymeric dye. Fluorescent polymeric dyes that find use in the subject methods and systems are varied. In some instances of the method, the polymeric dye includes a conjugated polymer. Conjugated polymers (CPs) are characterized by a delocalized electronic structure which includes a backbone of alternating unsaturated bonds (e.g., double and/or triple bonds) and saturated (e.g., single bonds) bonds, where π-electrons can move from one bond to the other. As such, the conjugated backbone may impart an extended linear structure on the polymeric dye, with limited bond angles between repeat units of the polymer. For example, proteins and nucleic acids, although also polymeric, in some cases do not form extended-rod structures but rather fold into higher-order three-dimensional shapes. In addition, CPs may form “rigid-rod” polymer backbones and experience a limited twist (e.g., torsion) angle between monomer repeat units along the polymer backbone chain. In some instances, the polymeric dye includes a CP that has a rigid rod structure. As summarized above, the structural characteristics of the polymeric dyes can have an effect on the fluorescence properties of the molecules.

Any convenient polymeric dye may be utilized in the subject methods and systems. In some instances, a polymeric dye is a multichromophore that has a structure capable of harvesting light to amplify the fluorescent output of a fluorophore. In some instances, the polymeric dye is capable of harvesting light and efficiently converting it to emitted light at a longer wavelength. In some cases, the polymeric dye has a light-harvesting multichromophore system that can efficiently transfer energy to nearby luminescent species (e.g., a “signaling chromophore”). Mechanisms for energy transfer include, for example, resonant energy transfer (e.g., Forster (or fluorescence) resonance energy transfer, FRET), quantum charge exchange (Dexter energy transfer) and the like. In some instances, these energy transfer mechanisms are relatively short range; that is, close proximity of the light harvesting multichromophore system to the signaling chromophore provides for efficient energy transfer. Under conditions for efficient energy transfer, amplification of the emission from the signaling chromophore occurs when the number of individual chromophores in the light harvesting multichromophore system is large; that is, the emission from the signaling chromophore is more intense when the incident light (the “excitation light”) is at a wavelength which is absorbed by the light harvesting multichromophore system than when the signaling chromophore is directly excited by the pump light.

The multichromophore may be a conjugated polymer. Conjugated polymers (CPs) are characterized by a delocalized electronic structure and can be used as highly responsive optical reporters for chemical and biological targets. Because the effective conjugation length is substantially shorter than the length of the polymer chain, the backbone contains a large number of conjugated segments in close proximity. Thus, conjugated polymers are efficient for light harvesting and enable optical amplification via Forster energy transfer.

Polymeric dyes of interest include, but are not limited to, those dyes described by Gaylord et al. in US Publication Nos. 20040142344, 20080293164, 20080064042, 20100136702, 20110256549, 20120028828, 20120252986 and 20130190193 the disclosures of which are herein incorporated by reference in their entirety; and Gaylord et al., J. Am. Chem. Soc., 2001, 123 (26), pp 6417-6418; Feng et al., Chem. Soc. Rev., 2010, 39, 2411-2419; and Traina et al., J. Am. Chem. Soc., 2011, 133 (32), pp 12600-12607, the disclosures of which are herein incorporated by reference in their entirety.

In some embodiments, the polymeric dye includes a conjugated polymer including a plurality of first optically active units forming a conjugated system, having a first absorption wavelength (e.g., as described herein) at which the first optically active units absorbs light to form an excited state. The conjugated polymer (CP) may be polycationic, polyanionic and/or a charge-neutral conjugated polymer.

The CPs may be water soluble for use in biological samples. Any convenient substituent groups may be included in the polymeric dyes to provide for increased water-solubility, such as a hydrophilic substituent group, e.g., a hydrophilic polymer, or a charged substituent group, e.g., groups that are positively or negatively charged in an aqueous solution, e.g., under physiological conditions. Any convenient water-soluble groups (WSGs) may be utilized in the subject light harvesting multichromophores. The term “water-soluble group” refers to a functional group that is well solvated in aqueous environments and that imparts improved water solubility to the molecules to which it is attached. In some embodiments, a WSG increases the solubility of the multichromophore in a predominantly aqueous solution (e.g., as described herein), as compared to a multichromophore which lacks the WSG. The water soluble groups may be any convenient hydrophilic group that is well solvated in aqueous environments. In some cases, the hydrophilic water soluble group is charged, e.g., positively or negatively charged. In certain cases, the hydrophilic water soluble group is a neutral hydrophilic group. In some embodiments, the WSG is a hydrophilic polymer, e.g., a polyethylene glycol, a cellulose, a chitosan, or a derivative thereof.

As used herein, the terms “polyethylene oxide”, “PEO”, “polyethylene glycol” and “PEG” are used interchangeably and refer to a polymer including a chain described by the formula —(CH₂—CH₂—O—)ₙ— or a derivative thereof. In some embodiments, “n” is 5000 or less, such as 1000 or less, 500 or less, 200 or less, 100 or less, 50 or less, 40 or less, 30 or less, 20 or less, 15 or less, such as 5 to 15, or 10 to 15. It is understood that the PEG polymer may be of any convenient length and may include a variety of terminal groups, including but not limited to, alkyl, aryl, hydroxyl, amino, acyl, acyloxy, and amido terminal groups. Functionalized PEGs that may be adapted for use in the subject multichromophores include those PEGs described by S. Zalipsky in “Functionalized poly(ethylene glycol) for preparation of biologically relevant conjugates”, Bioconjugate Chemistry 1995, 6 (2), 150-165. Water soluble groups of interest include, but are not limited to, carboxylate, phosphonate, phosphate, sulfonate, sulfate, sulfinate, ester, polyethylene glycols (PEG) and modified PEGs, hydroxyl, amine, ammonium, guanidinium, polyamine and sulfonium, polyalcohols, straight chain or cyclic saccharides, primary, secondary, tertiary, or quaternary amines and polyamines, phosphonate groups, phosphinate groups, ascorbate groups, glycols, including, polyethers, —COOM′, —SO₃M′, —PO₃M′, —NR₃⁺, Y′, (CH₂CH₂O)ₚR and mixtures thereof, where Y′ can be any halogen, sulfate, sulfonate, or oxygen containing anion, p can be 1 to 500, each R can be independently H or an alkyl (such as methyl) and M′ can be a cationic counterion or hydrogen, —(CH₂CH₂O)_{yy}CH₂CH₂XR^{yy}, —(CH₂CH₂O)_{yy}CH₂CH₂X—, —X(CH₂CH₂O)_{yy}CH₂CH₂—, glycol, and polyethylene glycol, wherein yy is selected from 1 to 1000, X is selected from 0, S, and NR^{ZZ}, and R^{ZZ} and R^{YY} are independently selected from H and C1-3 alkyl.

The polymeric dye may have any convenient length. In some cases, the particular number of monomeric repeat units or segments of the polymeric dye may fall within the range of 2 to 500,000, such as 2 to 100,000, 2 to 30,000, 2 to 10,000, 2 to 3,000 or 2 to 1,000 units or segments, or such as 100 to 100,000, 200 to 100,000, or 500 to 50,000 units or segments.

The polymeric dyes may be of any convenient molecular weight (MW). In some cases, the MW of the polymeric dye may be expressed as an average molecular weight. In some instances, the polymeric dye has an average molecular weight of from 500 to 500,000, such as from 1,000 to 100,000, from 2,000 to 100,000, from 10,000 to 100,000 or even an average molecular weight of from 50,000 to 100,000. In certain embodiments, the polymeric dye has an average molecular weight of 70,000.

In certain instances, the polymeric dye includes the following structure:

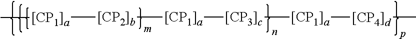

wherein CP₁, CP₂, CP₃ and CP₄ are independently a conjugated polymer segment or an oligomeric structure, wherein one or more of CP₁, CP₂, CP₃ and CP₄ are bandgap-lowering n-conjugated repeat units.

In some instances, the polymeric dye includes the following structure:

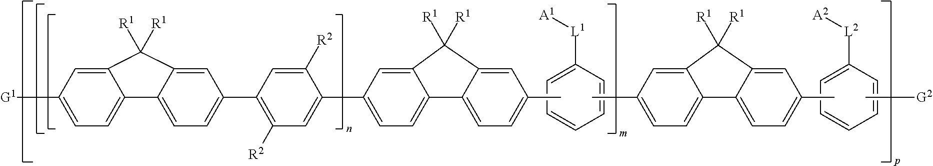

wherein each R¹ is independently a solubilizing group or a linker-dye; L¹ and L² are optional linkers; each R² is independently H or an aryl substituent; each A¹ and A² is independently H, an aryl substituent or a fluorophore; G¹ and G² are each independently selected from the group consisting of a terminal group, a π conjugated segment, a linker and a linked specific binding member; each n and each m are independently 0 or an integer from 1 to 10,000; and p is an integer from 1 to 100,000. Solubilizing groups of interest include alkyl, aryl and heterocycle groups further substituted with a hydrophilic group such as a polyethylglycol (e.g., a PEG of 2-20 units), an ammonium, a sulphonium, a phosphonium, and the like.

In some cases, the polymeric dye includes, as part of the polymeric backbone, a conjugated segment having one of the following structures:

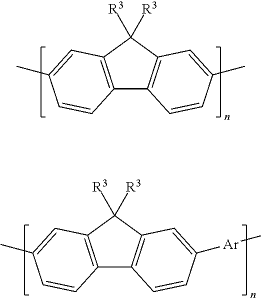

where each R³ is independently an optionally substituted alkyl or aryl group; Ar is an optionally substituted aryl or heteroaryl group; and n is 1 to 10000. In certain embodiments, R³ is an optionally substituted alkyl group. In certain embodiments, R³ is an optionally substituted aryl group. In some cases, R³ is substituted with a polyethyleneglycol, a dye, a chemoselective functional group or a specific binding moiety. In some cases, Ar is substituted with a polyethyleneglycol, a dye, a chemoselective functional group or a specific binding moiety.

In some instances, the polymeric dye includes the following structure:

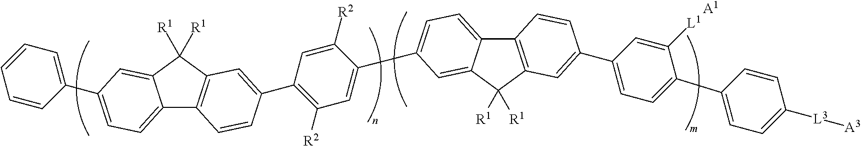

wherein each R¹ is a solubilizing group or a linker-dye group; each R² is independently H or an aryl substituent; L₁ and L₂ are optional linkers; each A¹ and A³ are independently H, a fluorophore, a functional group or a specific binding moiety (e.g., an antibody); and n and m are each independently 0 to 10000, wherein n+m>1.

The polymeric dye may have one or more desirable spectroscopic properties, such as a particular absorption maximum wavelength, a particular emission maximum wavelength, extinction coefficient, quantum yield, and the like (see e.g., Chattopadhyay et al., “Brilliant violet fluorophores: A new class of ultrabright fluorescent compounds for immunofluorescence experiments.” Cytometry Part A, 81A(6), 456-466, 2012).

In some embodiments, the polymeric dye has an absorption curve between 280 and 475 nm. In certain embodiments, the polymeric dye has an absorption maximum in the range 280 and 475 nm. In some embodiments, the polymeric dye absorbs incident light having a wavelength in the range between 280 and 475 nm. In some embodiments, the polymeric dye has an emission maximum wavelength ranging from 400 to 850 nm, such as 415 to 800 nm, where specific examples of emission maxima of interest include, but are not limited to: 421 nm, 510 nm, 570 nm, 602 nm, 650 nm, 711 nm and 786 nm. In some instances, the polymeric dye has an emission maximum wavelength in a range selected from the group consisting of 410-430 nm, 500-520 nm, 560-580 nm, 590-610 nm, 640-660 nm, 700-720 nm and 775-795 nm. In certain embodiments, the polymeric dye has an emission maximum wavelength of 421 nm. In some instances, the polymeric dye has an emission maximum wavelength of 510 nm. In some cases, the polymeric dye has an emission maximum wavelength of 570 nm. In certain embodiments, the polymeric dye has an emission maximum wavelength of 602 nm. In some instances, the polymeric dye has an emission maximum wavelength of 650 nm. In certain cases, the polymeric dye has an emission maximum wavelength of 711 nm. In some embodiments, the polymeric dye has an emission maximum wavelength of 786 nm. In certain instances, the polymeric dye has an emission maximum wavelength of 421 nm±5 nm. In some embodiments, the polymeric dye has an emission maximum wavelength of 510 nm±5 nm. In certain instances, the polymeric dye has an emission maximum wavelength of 570 nm±5 nm. In some instances, the polymeric dye has an emission maximum wavelength of 602 nm±5 nm. In some embodiments, the polymeric dye has an emission maximum wavelength of 650 nm±5 nm. In certain instances, the polymeric dye has an emission maximum wavelength of 711 nm±5 nm. In some cases, the polymeric dye has an emission maximum wavelength of 786 nm±5 nm. In certain embodiments, the polymeric dye has an emission maximum selected from the group consisting of 421 nm, 510 nm, 570 nm, 602 nm, 650 nm, 711 nm and 786 nm.

In some instances, the polymeric dye has an extinction coefficient of 1×10⁶ cm⁻¹M⁻¹ or more, such as 2×10⁶ cm⁻¹M⁻¹ or more, 2.5×10⁶ cm⁻¹M⁻¹ or more, 3×10⁶ cm⁻¹M⁻¹ or more, 4×10⁶ cm⁻¹M⁻¹ or more, 5×10⁶ cm⁻¹M⁻¹ or more, 6×10⁶ cm⁻¹M⁻¹ or more, 7×10⁶ cm⁻¹M⁻¹ or more, or 8×10⁶ cm⁻¹M⁻¹ or more. In certain embodiments, the polymeric dye has a quantum yield of 0.05 or more, such as 0.1 or more, 0.15 or more, 0.2 or more, 0.25 or more, 0.3 or more, 0.35 or more, 0.4 or more, 0.45 or more, 0.5 or more, or even more. In certain cases, the polymeric dye has a quantum yield of 0.1 or more. In certain cases, the polymeric dye has a quantum yield of 0.3 or more. In certain cases, the polymeric dye has a quantum yield of 0.5 or more. In some embodiments, the polymeric dye has an extinction coefficient of 1×10⁶ or more and a quantum yield of 0.3 or more. In some embodiments, the polymeric dye has an extinction coefficient of 2×10⁶ or more and a quantum yield of 0.5 or more.

In some instances, the fluorescently labeled chlorotoxin binding member is one of the fluorescently labeled chlorotoxin binding members described in published PCT application publication no. WO/2015/042202, the disclosure of which fluorescently labeled chlorotoxin binding members are herein incorporated by reference.

In producing the fluorescently labeled blood sample, the blood sample is contacted with the fluorescently labeled chlorotoxin binding member using any convenient protocol. The amount of fluorescently labeled chlorotoxin binding member that is combined with the blood sample in the contacting step may vary, wherein in some instances the amount is sufficient to provide a concentration in the labeled sample of 100 μM to 10 nM. The fluorescently labeled chlorotoxin binding member and the sample may be contacted using a mixing protocol, e.g., that employs agitation, stirring, etc. as desired. The temperature may vary, ranging in some instances from 4 to 37° C. The incubation time may vary, ranging in some instances from 5 minute to 20 hours, such as 15 minutes to 15 hours, e.g., 30 minutes to 12 hours, including 30 minutes to 6 hours.

The fluorescently labeled blood sample may be produced as the same location as the location where it is cytometrically assayed (which may also be the location of sample obtainment), or at a location that is different from the location of cytometric assaying, where this different location may or may not be the location of sample obtainment, i.e., it may be a remote location from the location of sample obtainment and/or cytometric assaying. By “remote location,” it is meant a location other than the location at which the sample obtainment and/or assaying occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. Where one or more of the sample obtainment, labeled sample production and cytometric assaying occurs at locations remote from the other, the methods further include receiving a sample and/or labeled sample from the remote location, depending on the embodiment. As such, in some instances the methods may include receiving the fluorescently labeled blood sample from a remote location. In those instances where the fluorescently labeled blood sample is present in a container, e.g., as described below, the method further includes removing the fluorescently labeled blood sample from the container.

As summarized above, aspects of the methods include cytometrically assaying the labeled sample to determine whether a CTC is present in the sample. As such, methods of the present disclosure are cytometric methods for the detection of CTCs in a sample. The term “cytometric methods” is used herein to describe flow cytometric methods and/or imaging cytometric methods. Accordingly, “cytometric assay” may refer to a flow cytometric assay and/or imaging cytometric assay, and “cytometer” may refer to a flow cytometer and/or imaging cytometer.

In some embodiments, methods of the invention of detecting a CTC in a sample are qualitative, where the detection of the CTC is qualitative, e.g., the determination is made that the CTC is or is not present in the sample. In some embodiments, methods of the invention of detecting a CTC in a sample are quantitative, where the detection of the CTC is quantitative. The methods can include determining a quantitative measure of the number of CTCs in a sample. In some embodiments, quantifying the number of CTCs in a sample includes determining whether the number of rare target cells present is above or below a predetermined threshold.

Aspects of embodiments of the invention include flow cytometrically assaying the labeled sample. In such embodiments, detecting a CTC in a flow cytometer may include exciting the fluorescent label of the fluorescently labeled chlorotoxin binding member with one or more lasers at an interrogation point of the flow cytometer, and subsequently detecting fluorescence emission from the label using one or more optical detectors, e.g., as described in greater detail below. It may be desirable, in addition to detecting the CTC, to determine the number of CTCs and/or sorting the CTCs. Accordingly, in one embodiment, the methods further include processing the sample (e.g., counting, sorting, or counting and sorting the CTCs and/or other cells of interest) by flow cytometry.

In detecting, counting and/or sorting CTCs/other cells, the labeled sample is introduced into the flow path of the flow cytometer. When in the flow path, the cells are passed substantially one at a time through one or more sensing regions (e.g., an interrogation point), where each of the cells in the sample is exposed individually to a source of light at a single wavelength and measurements of light scatter parameters and/or fluorescent emissions as desired (e.g., two or more light scatter parameters and measurements of one or more fluorescent emissions) are separately recorded for each cell. The data recorded for each cell is analyzed in real time or stored in a data storage and analysis means, such as a computer, as desired. U.S. Pat. No. 4,284,412 describes the configuration and use of a typical flow cytometer equipped with a single light source, while U.S. Pat. No. 4,727,020 describes the configuration and use of a flow cytometer equipped with two light sources. The disclosures of these patents are herein incorporated by reference in their entireties for all purposes. Flow cytometers having more than two light sources may also be employed.

More specifically, in a flow cytometer, the cells of the sample are passed, in suspension, substantially one at a time in a flow path through one or more sensing regions (or “interrogation points”) where in each region each cell is illuminated by an energy source. The energy source may include an illuminator that emits light of a single wavelength, such as that provided by a laser (e.g., He/Ne or argon) or a mercury arc lamp with appropriate filters. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 405 nm, 535 nm, 635 nm, and the like.

In series with a sensing region, detectors, e.g., light collectors, such as photomultiplier tubes (or “PMT”), are used to record light that passes through each analyte (generally referred to as forward light scatter), light that is reflected orthogonal to the direction of the flow of the analytes through the sensing region (generally referred to as orthogonal or side light scatter) and fluorescent light emitted from the labeled analyte, as the analyte passes through the sensing region and is illuminated by the energy source. Each of forward light scatter (or FSC), orthogonal light scatter (SSC), and fluorescence emissions (FL1, FL2, etc.) comprise a separate parameter for each analyte (or each “event”). Thus, for example, two, three or four parameters can be collected (and recorded) from a cell labeled with two different fluorescent labels.

Accordingly, in flow cytometrically assaying cells, cells may be detected and uniquely identified by exposing the particles to excitation light and measuring the fluorescence of each cell in one or more detection channels, as desired. The excitation light may be from one or more light sources and may be either narrow or broadband. Examples of excitation light sources include lasers, light emitting diodes, and arc lamps. Fluorescence emitted in detection channels used to identify the cells may be measured following excitation with a single light source, or may be measured separately following excitation with distinct light sources. If separate excitation light sources are used to excite the fluorescent labels, the labels may be selected such that all the labels are excitable by each of the excitation light sources used.

Flow cytometers further include data acquisition, analysis and recording components, such as a computer, wherein multiple data channels record data from each detector for the light scatter and fluorescence emitted by each cell as it passes through the sensing region. The purpose of the analysis system is to classify and count cells where each cell presents itself as a set of digitized parameter values. In flow cytometrically assaying (e.g., detecting, counting and/or sorting) cells/particles in methods of the present disclosure, the flow cytometer may be set to trigger on a selected parameter in order to distinguish the cells of interest from background and noise. “Trigger” refers to a preset threshold for detection of a parameter. It is typically used as a means for detecting passage of a particle through the laser beam. Detection of an event which exceeds the threshold for the selected parameter triggers acquisition of light scatter and fluorescence data for the cell. Data is not acquired for cells or other components in the sample being assayed which cause a response below the threshold. The trigger parameter may be the detection of forward scattered light caused by passage of cell through the light beam. The flow cytometer then detects and collects the light scatter and fluorescence data for the cell.

A particular subpopulation of interest is then further analyzed by “gating” based on the data collected for the entire population. To select an appropriate gate, the data is plotted so as to obtain the best separation of subpopulations possible. This procedure is typically done by plotting forward light scatter (FSC) vs. side (i.e., orthogonal) light scatter (SSC) on a two dimensional dot plot. The flow cytometer operator then selects the desired subpopulation of cells (i.e., those cells within the gate) and excludes cells which are not within the gate. Where desired, the operator may select the gate by drawing a line around the desired subpopulation using a cursor on a computer screen. Only those cells within the gate are then further analyzed by plotting the other parameters for these cells, such as fluorescence.

Flow cytometric assay procedures are further described in the art. See, e.g., Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49(pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain aspects, flow cytometrically assaying the sample involves using an analyzing flow cytometer, such as but not limited to: the BD Accuri™ C6 flow cytometer; the BD FACSCanto™ II flow cytometer; the BD FACSVerse™ flow cytometer; the BD LSRFortessa™ flow cytometer; the BD LSRFortessa™ X-20 flow cytomter; etc. Methods of the present disclosure may involve image cytometry, such as is described in Holden et al. (2005) Nature Methods 2:773 and Valet, et al. 2004 Cytometry 59:167-171, the disclosures of which are incorporated herein by reference.

Cytometric analysis may include sorting. Some flow cytometers are equipped to sort particles as they flow through the machine, redirecting the particle (after the particle has been interrogated/evaluated) to a particular location (e.g., into a desired sample collection container). During sorting, the fluid stream is broken into highly uniform droplets, which detach from the stream. The time between when a particle intercepts the energy source (e.g., the laser) and when it reaches the droplet breakoff point is determined. When a particle is detected that meets the predefined sorting criteria, an electrical charge is applied to the stream just as the droplet containing that particle breaks off from the stream. Once broken off from the stream, the droplet—now surrounded by air—retains its charge. The charged droplet passes by two strongly charged deflection plates. Electrostatic attraction and repulsion cause each charged droplet to be deflected to the left or right, depending on the droplet's charge polarity. For example, in some cases, a flow cytometer can sort particles into one of two different tubes, or into a desired well of a multi-well plate (e.g., a 6-well plate, a 12-well plate, a 24-well plate, a 48-well plate, a 96-well plate, etc.). Uncharged droplets are not affected by the electric field and pass down the center to be collected or aspirated as waste.

Where the cells are sorted, any convenient flow cytometer having sorting capability may be employed. Examples of sorting flow cytometers that may be employed include, but are not limited to: the BD FACSAria™ Fusion cell sorter; the BD FACSJazz™ cell sorter; the BD FACSAria™ III cell sorter; the BD Influx™ cell sorter; etc.

Where desired, CTCs may be sorted and subsequently analyzed by any convenient analysis technique. Subsequent analysis techniques of interest include, but are not limited to, sequencing; assaying by CellSearch, as described in Food and Drug Administration (2004) Final rule. Fed Regist 69: 26036-26038; assaying by CTC Chip, as described in Nagrath, et al. (2007) Nature 450: 1235-1239; assaying by MagSweeper, as described in Talasaz, et al. (2009). Proc Natl Acad Sci USA 106: 3970-3975; and assaying by nanostructured substrates, as described in Wang S, et al. (2011) Angew Chem Int Ed Engl 50: 3084-3088; the disclosures of which are incorporated herein by reference. Where desired, the sorting protocol may include distinguishing viable and dead rare cells, where any convenient staining protocol for identifying such cells may be incorporated in to the methods.

In certain aspects, non-rare cells may be separated from a sample prior to cytometric analysis. Any convenient means of removing non-rare cells from a sample may be employed. Separation methods of interest include, but are not limited to, magnetic separation techniques, such as those described in U.S. Pat. Nos. 5,945,281, 6,858,440; 6,645,777; 6,630,355; and 6,254,830; US Patent Application No. PCT/US2012/032423; and Hoeppener, et al. (2012) Recent Results Cancer Res. 195:43-58; the disclosures of which are incorporated herein by reference. Separation methods of interest further include those comprising acoustic concentrators or separators, such as those described in U.S. Pat. No. 6,929,750, the disclosure of which is hereby incorporated by reference.

In certain embodiments the source of the sample is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

### Systems

Also provided are cytometric systems for practicing the subject methods. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, data input devices, e.g., interface ports, a mouse, a keyboard, etc., fluid handling components, power sources, etc.

In certain aspects, a cytometric system includes a cytometric sample fluidic subsystem configured to contact a sample with a fluorescently labeled chlorotoxin binding member, e.g., as described. Systems may include a cytometer fluidically coupled to the cytometric sample fluidic subsystem. In other aspects, systems may include a cytometric sample fluidic subsystem configured to contact a sample with a fluorescently labeled chlorotoxin binding member and a cytometer fluidically coupled to the flow cytometric sample fluidic subsystem, the cytometer configured to assay the sample for the presence of cells bound by the fluorescently labeled chlorotoxin binding member. In certain aspects, the cytometer is configured to use the emission signal from the fluorescently labeled chlorotoxing binding member as a detection threshold. As such, aspects of the invention include cytometric systems, such as flow cytometric systems, comprised of a blood sample labeled with a fluorescently labeled chlorotoxin binding member, e.g., as described above.

### Utility

The subject methods and compositions find use in a variety of applications including, but not limited to, the detection and/or isolation of circulating tumor cells. The enumeration and characterization of circulating tumor cells (CTCs) in blood has been proposed as a real-time clinical biomarker that is more sensitive and less invasive than alternative methods of cancer diagnostic, prognostic, and pharmacological applications. However, CTCs are rare and difficult to distinguish from abundant leukocytes, making their isolation a major technological challenge. The subject methods and compositions facilitate detection and/or isolation of CTCs, which may then be further analyzed for diagnostic and/or research purposes.

The presence of CTCs is associated with decreased cancer survival rates and their continued presence during therapy indicates that an alternative therapy should be considered in patients with metastatic and even localized cancer. Detections of CTCs according to the invention may be employed in methods of selecting optimal therapy for individual patients during the course of cancer treatment.

In some instances, the methods are performed without intracellular staining, e.g., as required in CTC detection methods that detect cytokeratins, and/or without amplification, e.g., as required in CTC detection methods that detect EpCAM.

### Containers

An aspect of the present disclosure includes a container for processing a blood sample for use in methods of the invention. Containers as described herein are useful for performing the steps of labeling a blood sample in the same container, thereby providing a processed blood sample, which may then be further analyzed, as desired.

Containers of interest may be fabricated from any convenient material. The container can be made of glass, plastic or other suitable materials. Plastic materials can be oxygen impermeable materials or contain an oxygen impermeable layer. Transparent materials are of interest, such as transparent thermoplastic materials like polycarbonates, polyethylene, polypropylene, polyethylene-terephtalate. The container also has a suitable dimension selected according to the required volume of the biological sample being collected. In one embodiment, containers have a tubular shape with an axial length of 60-mm to 130-mm and a diameter of 10-mm to 20-mm. A container that is a tube having an axial length ranging from 75-mm and 100-mm millimeters and a diameter ranging 13-mm to 16-mm is of interest.

The containers may include a closure material over an otherwise opening of the container material. In some instances, the closure member is made of a resilient material to provide a seal for retaining the sample in the container. Of interest is a closure made of a resilient material capable of maintaining an internal pressure differential less than atmospheric and that can be pierced by a needle to introduce a biological sample into the container. Suitable materials for closure include, for example, silicone rubber, natural rubber, styrene butadiene rubber, ethylene-propylene copolymers and polychloroprene. Thus in certain embodiments, the closure member is a septum pierceable by a cannula. The closure may also provide convenient access to a biological sample within the container, as well as a protective shield that overlies the closure. As such, the closure may further include a removable cover, such as a threaded or snap-on cap or other suitable member that can be applied over the outside of the closure for various purposes. For instance, a threaded cap can be screwed over the closure after the sample collection to provide a second seal and further increase user safety. Any component of the device can be color coded, labeled, or otherwise tagged or marked for easy identification.

The device as assembled can be provided to maintain an internal pressure differential between atmospheric pressure outside of the container and is at a pressure less than atmospheric pressure. The pressure can be selected to draw a predetermined volume of a biological sample. In some instances, the biological sample is drawn into the first chamber by piercing a closure comprising a resilient material with a needle or cannula, such as is typical for known evacuated sample containers for drawing blood. An example of a suitable containers and closures are disclosed in U.S. Pat. No. 5,860,937, which reference is incorporated in its entirety. One aspect of interest is where the internal pressure of the container is selected to draw a predetermined volume of about 2.0 ml to about 10 ml of biological sample into the first chamber, and more particularly, about 2.5 ml to about 5 ml into the first chamber of the device.

As such, containers of interest may enclose an evacuated interior volume having a fluorescently labeled chlorotoxin binding member, e.g., described above, present therein. In certain embodiments, the container may have an elongated shape where the length of the container is greater than a cross-section of an opening of the container. Embodiments of containers of interest include containers shaped in a form of a tube. The tube may have a flat bottom, conical bottom, or a rounded bottom. In certain cases, the container may enclose an evacuated interior volume defined by a bottom wall and a side wall, the side wall further defining an open end. The volume of the container may vary as desired, and in some instances ranges from 1 to 1000 ml, such as 5 to 500 ml, e.g., 5 to 100 ml, including 5 to 50 ml. The open end or ends of the container may be sealed with sealing elements, as desired, such as by a septum, e.g., where an opening is covered by a puncturable septum.

As noted above, a fluorescently labeled chlorotoxin binding member may be disposed inside the container. In certain embodiments, the fluorescently labeled chlorotoxin binding member may be coated on the interior surface, e.g., a bottom wall and/or a side wall, of the container. In some embodiments, the coating of the fluorescently labeled chlorotoxin binding member is dried on the interior surface of the container. In some cases, the fluorescently labeled chlorotoxin binding member is stably associated, e.g., covalenty attached, to an interior wall of the container. In certain cases, the fluorescently labeled chlorotoxin binding member may be disposed in the container in a liquid form. The container may be provided with the fluorescently labeled chlorotoxin binding member in a liquid or the liquid may be subsequently dried and the container may be provided with the fluorescently labeled chlorotoxin binding member in a dried form, e.g., lyophilized form.

In certain cases, the container may include additives to improve stability of the fluorescently labeled chlorotoxin binding member, such as buffers, glycerol, or phenylmethanesulfonyl fluoride (PMSF). Additional additives may also be present in the container, such as, additives that preserve the cells present in whole blood, e.g., platelet stabilizing factor, and the like. Exemplary additives that may be included in the container are anticoagulants such as ethylenediaminetetraacetic acid (EDTA), buffered citrate, or heparin. The container may include these additives in a liquid or dried state. In certain cases, the container may be coded to facilitate identification and/or tracking of the container. Any component of the container can be color coded, labeled, or otherwise tagged or marked for easy identification.

As noted above, the container is evacuated, i.e., the interior of the container is at a pressure less than atmospheric pressure. The pressure may be selected to draw a predetermined volume of a whole blood sample. For example, the container may draw a whole blood sample of 1 ml-20 ml, e.g., 2 ml-10 ml, such as, 2 ml, 3 ml, 4 ml, 5 ml, 6 ml, 7 ml, 8 ml, 9 ml, or 10 ml.

In some instances, the whole blood sample is drawn into the container by piercing the septum with a needle or cannula, such as is typical for known evacuated sample containers for drawing blood. Examples of suitable containers and closures are disclosed in U.S. Pat. Nos. 5,860,937; 5,344,611; 5,326,535; 5,320,812; 5,257,633 and 5,246,666, each of which is incorporated by reference here in its entirety.

### Kits

Also provided are kits for practicing one or more embodiments of the above-described methods. The subject kits may include various components and reagents. In some instances, the kits include at least reagents finding use in the methods (e.g., as described above, such as a fluorescently labeled chlorotoxin binding member and/or blood sample collection container that includes the same, e.g., as described above); and/or a computer readable medium having a computer program stored thereon, wherein the computer program, when loaded into a computer, operates the computer to perform a cytometric assay as described herein; and a physical substrate having an address from which to obtain the computer program.

In addition to the above components, the subject kits may further include instructions for practicing the methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., CD, DVD, Blu-Ray, flash memory, etc., on which the information has been recorded. Yet another means that may be present is a website address which may be used via the Internet to access the information at a removed site. Any convenient means may be present in the kits.

The following examples are offered by way of illustration and not by way of limitation.

### EXPERIMENTAL

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### A. Materials and Methods

Chlorotoxin:Cy5.5 as described in Veiseh et al., “Tumor paint: a chlorotoxin:cy5.5 bioconjugate for intraoperative visualization of cancer foci,” Cancer Res. (2007) 67:6882-6888 is employed as a fluorescent label.

Blood samples are obtained and labeled as described below.

All flow cytometric assays are performed using a BD Biosciences FACSCanto™ flow cytometer. All reagents and materials are used following manufacturer's protocols.

### B. Detection of Tumor Cells in Blood Samples

Venous blood of normal donors is collected in sodium heparin BD Vacutainer tubes. HT-29 tumor cells are added to blood samples at concentrations of (i) 10,000 HT-29 cells/mL; (ii) 1,000 HT-29 cells/mL; (iii) 100 HT-29 cells/mL; or (iv) 0 HT-29 cells/mL. For each of the four samples, a 7.5 mL draw is taken and spun in a BD Vacutainer CPT tube to separate the WBC portion. The WBC fraction is subsequently stained with Chlorotoxin:Cy5.5 and analyzed via FACS.

The resulting density plots show a population of cells in the samples where HT-29 tumor cells had starting concentrations of (i) 10,000 HT-29 cells/mL; (ii) 1,000 HT-29 cells/mL; and 100 HT-29 cells/mL. The number of cells observed in each plot decreases in proportion to the starting concentration of the HT-29 tumor cells. No cells are observed in where the starting HT-29 tumor cell concentration was 0 cells/mL.

Although the foregoing invention has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of determining whether a circulating tumor cell (CTC) marker is present in a sample, the method comprising:
flow cytometrically assaying a fluorescently labeled sample that has been fluorescently labeled with a fluorescently labeled chlorotoxin binding member to determine whether a CTC marker is present in the blood sample.

2. The method according to claim 1, wherein the CTC marker is a CTC.

3. The method according to claim 1, wherein the sample is a blood sample.

4. The method according to claim 1, wherein the fluorescently labeled chlorotoxin binding member comprises a chlorotoxin or mutant thereof.

5-7. (canceled)

8. The method according to claim 1, wherein the fluorescently labeled chlorotoxin comprises a fluorescent dye having an excitation maximum ranging from 300 to 700 nm and an emission maximum ranging from 400 to 800 nm.

9. The method according to claim 8, wherein the fluorescent dye comprises a cyanine dye.

10. The method according to claim 9, wherein the cyanine dye is Cy5.5.

11-12. (canceled)

13. The method according to claim 1, wherein the sample comprises a mammalian sample.

14. The method according to claim 13, wherein the mammalian sample comprises a human blood sample.

15. The method according to claim 1, the method further comprising contacting a sample with a fluorescently labeled chlorotoxin binding member to produce the fluorescently labeled sample.

16. The method according to claim 1, the method further comprising receiving the fluorescently labeled sample from a remote location.

17. The method according to claim 16, wherein the fluorescently labeled sample is present in a container and the method comprises removing the fluorescently labeled sample from the container.

18. The method according to claim 1, wherein flow cytometrically assaying the sample comprises counting CTCs.

19. The method according to claim 1, wherein flow cytometrically assaying the sample comprises sorting CTCs.

20. The method according to claim 19, wherein the method further comprises collecting detected CTCs.

21. The method according to claim 20, wherein the method further comprises assaying a collected CTC.

22. A fluorescently labeled sample that has been fluorescently labeled with a fluorescently labeled chlorotoxin binding member.

23-31. (canceled)

32. The fluorescently labeled sample according to claim 22, wherein the sample comprises a mammalian blood sample.

33. The fluorescently labeled sample according to claim 32, wherein the mammalian blood sample comprises a human blood sample.

34. (canceled)

35. A kit for identifying a CTC marker in a sample, the kit comprising:
a container configured to receive an amount of a sample; and
a fluorescently labeled chlorotoxin binding member.

36-42. (canceled)


SEQUENCE LISTING (4 sequences)

SEQ ID NO: 1 - protein, 36 aa, Leiurus quinquestriatus
MCMPCFTTDHQMARKCDDCCGGKGRGKCYGPQCLCR

SEQ ID NO: 2 - protein, 36 aa, Artificial Sequence
  Synthetic polypeptide
MCMPCFTTDHQMARRCDDCCGGRGRGKCYGPQCLCR

SEQ ID NO: 3 - protein, 33 aa, Buthus martensii Karsch
CGPCFTTDANMARKCRECCGGIGKCFGPQCLCN

SEQ ID NO: 4 - protein, 34 aa, Androctonus australis
MCIPCFTTNPNMAAKCNACCGSRRGSCRGPQCIC